(19) United States
(12) Patent Application Publication
(10) Pub. No.: US 2023/0178226 A1
(43) Pub. Date: Jun. 8, 2023

(54) System and Method for Validating a System and Method for Monitoring Pharmaceutical Operations

(71) Applicants: Fresenius Kabi Austria GmbH, Graz (AT); KML Vision GmbH, Graz (AT)

(72) Inventors: Christoph Köth, Graz (AT); Martin Kleinhenn, Gratkorn (AT); Philipp Kainz, Graz (AT); Andrea Maffeis, St. Josef (AT); Michael Mayrhofer-Reinhartshuber, Thalgau (AT); Thomas Ebner, Saalfelden (AT); Christina Egger, Graz (AT)

(21) Appl. No.: 18/061,976

(22) Filed: Dec. 5, 2022

(30) Foreign Application Priority Data
Dec. 6, 2021 (EP) 21212628.8

(51) Int. Cl. G16H 40/20; G16H 20/10; G06V 10/764; G06V 10/50

(52) U.S. Cl. CPC G16H 40/20; G16H 20/10; G06V 10/764; G06V 10/50

(57) ABSTRACT

A system for validating a system for monitoring pharmaceutical operations includes at least one controller configured to perform a classification of an intervention captured by one or more image frames using a classification model, the classification model being trained with image frames of interventions assigned to at least two different classes. The at least one controller is also configured to assign a value to an individual feature of a feature set associated with the classification, the value corresponding to a contribution of the individual feature to the classification. In addition, the at least one controller is configured to generate a graphical representation of the values for the features contributing to the classification of the interventions.

### CROSS REFERENCE TO RELATED APPLICATIONS

The present application claims priority to European Patent App. No. 21212628.8, filed Dec. 6, 2021, which is hereby incorporated herein by reference in its entirety.

This patent is directed to a system and method for validating a system and method for monitoring pharmaceutical operations. In particular, this patent is directed to a system and method for validating a system and method for monitoring pharmaceutical operations that relies on artificial intelligence (AI).

Many pharmaceutical operations, for example in an aseptic pharma production, have to be performed in a sterile environment typically provided by an isolator or similar system providing, e.g., clean room class A. Filling operations in particular are often critical, as may be cleaning operations. All interventions, even when performed with glove protection, can negatively affect product sterility and are thus typically closely monitored, documented, and analyzed for a potential impact.

It is common practice to use equipment, such as light barriers, to automatically detect interventions, e.g., when a human operator uses a glove in an isolator. This detection occurs as a safety measure for the operator. It is not possible to distinguish between different classes of interventions, e.g., between critical interventions and non-critical interventions, relative to the pharma product.

It is believed that the use of artificial intelligence (AI) techniques (which could utilize machine learning (ML) or deep learning (DL)) could provide a system and method that is capable of distinguishing between different classes of interventions in a reliable and repeatable manner. One challenge to be overcome in the adoption of such a system is the need to provide validation of the system and method sufficient to the standards of the industry.

To begin, it will be recognized that the validation standards of the pharmaceutical industry are relatively high. That is, high-risk and/or sensitive industries that are highly regulated have higher standards for quality assurance, validation, and/or auditing when adopting new technologies. The pharmaceutical sector or industry is one such industry where higher standards exist.

Moreover, there is a tension between AI systems and ML models and conventional systems and methods for validation and auditing. While interpretable ML models are (to some degree) directly comprehensible, many AI systems rely on non-transparent ML models, i.e., black box models. In addition, the AI systems and ML models are becoming increasingly more complex. The rising complexity makes it more difficult to understand the underlying reasoning of the AI system or ML model. This limited understanding of the decision process presents issues when attempting to validate and/or audit the system and method.

Further, known instances of biased AI systems increase the level of caution. Alternatively, such biased AI systems decrease the level of trust. The use of AI systems in sensitive areas can have major consequences when decisions are based on biased data or wrong decision criteria, and the level of caution or trust only exacerbates perceived level of risk.

### SUMMARY

According to one aspect of the present disclosure, a system for validating a system for monitoring pharmaceutical operations includes at least one controller configured to perform a classification of an intervention captured by one or more image frames using a classification model, the classification model being trained with image frames of interventions assigned to at least two different classes. The at least one controller is also configured to assign a value to an individual feature of a feature set associated with the classification, the value corresponding to a contribution of the individual feature to the classification. In addition, the at least one controller is configured to generate a graphical representation of the values for the features contributing to the classification of the interventions.

According to another aspect of the present disclosure, a method for validating a method for monitoring pharmaceutical operations includes performing a classification of an intervention captured by one or more image frames using a classification model, the classification model being trained with image frames of interventions assigned to at least two different classes. The method also includes assigning a value to an individual feature of a feature set associated with the classification, the value corresponding to a contribution of the individual feature to the classification. In addition, the method includes generating a graphical representation of the values for the features contributing to classification of the interventions.

### BRIEF DESCRIPTION OF DRAWINGS

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings is necessarily to scale.

FIG. 1 shows a flowchart for an embodiment of a validation approach for a system and method for monitoring pharmaceutical operations.

FIG. 2 shows a flowchart for another embodiment of a validation approach for a system and method for monitoring pharmaceutical operations.

FIG. 3 shows a system for monitoring critical pharmaceutical operations in an aseptic interior space using two cameras and first and second models, such as may be validated by the approach of FIG. 1 or FIG. 2;

FIG. 4 shows an image frame assembled from images taken by the two cameras, showing the interior space;

FIG. 5 shows a method for monitoring critical pharmaceutical operations in an aseptic interior space using two cameras and first and second models;

FIG. 6 shows a video stream comprising several image frames, and a difference image computed based on a reference frame and a current frame;

FIG. 7 shows parts of image frames of a critical intervention recorded by the two cameras, a respective difference image, respective difference images and histograms of oriented gradients; and

FIG. 8 shows parts of images of a non-critical intervention recorded by the two cameras, a respective difference image, respective difference images and histograms of oriented gradients.

### DETAILED DESCRIPTION OF VARIOUS EMBODIMENTS

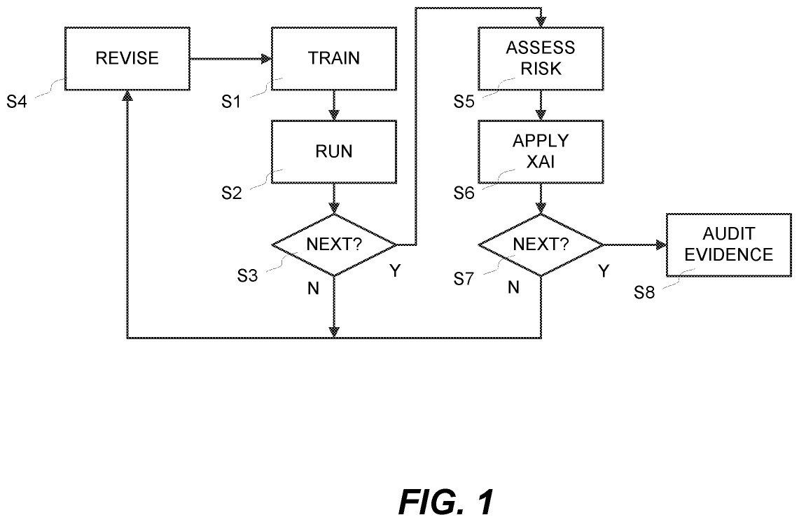

FIG. 1 is an embodiment of a method for validating a system and method for monitoring pharmaceutical operations, and according to some embodiments, for monitoring pharmaceutical operations in critical areas or in geometrically defined areas. It may also be useful for validating a system and method for monitoring critical pharmaceutical operations, which may include the production of medicine or medical nutrition or the like.

The method may be carried out using at least one controller configured to perform the actions (steps) of the method. All of the actions of the method may be performed by a single controller. In other embodiments, certain of the actions may be performed by a first controller, and other actions may be performed by a second (or a third, etc.) controller. For example, the system for monitoring pharmaceutical operations may include a first controller for carrying out steps related to the monitoring, and the system for validating may include a second controller that is in communication with the first controller, either directly or indirectly, as well as the first controller.

The controller may be defined by one or more electrical circuit components. According to other embodiments, one or more processors (or simply, the processor) may be programmed to perform the actions of the controller according to an executable code stored in a memory. According to still further embodiments, the controller may be defined in part by electrical circuit components and in part by a processor programmed to perform the actions of the controller.

As shown in FIG. 1, the method may begin with the step S1. Prior to this step (the set-up phase), a new AI system may have been considered. A use case definition may be prepared, an expert team formed, and an initial risk assessment made. The use case definition may include establishing the initial requirements for the AI system. Based on the use case definition, the expert team is formed, and may include domain experts, end-users, and quality assurance, project management, and technology suppliers. The initial risk assessment is conducted by the expert team, and may cover business and quality issues, technical and AI specific risks, risks to the end-user, and risks to society.

At step S1, the system specifications are combined with the training data set to train the model. The step of compilation of the training data set concurs with the elaboration of the system specifications. The scheme for compiling the training data set should consider traditional data issues such as correct distribution, labelling, potential biases, completeness, and sources. The scheme should also consider domain specific issues that may arise in the context in which the AI system is to be used.

For example, the model may be a model that performs a classification of an intervention captured by one or more image frames. The model, which may be referred to as a classification model, may be trained with image frames of interventions assigned to at least two different classes. For example, there may be two classes, critical and non-critical. According to other embodiments, the classification model may classify an operation as startup, cleaning, filling, or compounding. Consequently, the model is not limited to these classes, or to only two classes.

After the model has been trained, the method moves to step S2, where the model is run to carry out its assigned task. The test data set should represent the real-life challenges the AI system is intended to address. It should include numerous scenarios that are likely to occur under normal conditions. Returning to the above example, the classification model may be used to perform a classification of an intervention captured by one or more image frames. The results of step S2 may be considered at step S3 as to whether the performance of the artificial intelligence (AI) system has identifiable limitations (e.g., does not meet predefined acceptance criteria), or if it is possible to continue with the method.

If limitations are identified at step S3, then the method passes to step S4, where the model is revised. This revision may take different forms. According to one form, the model may require additional training with additional image frames. The training may include additional image frames assigned to the at least two different classes. The training may include entirely new sets of image frames. The training may include image frames with revised labeling, for example to reduce the label noise. Revision may also involve changing specifications (e.g., model architecture and hyperparameters (such as training time, sampling strategies, randomizations, data augmentation, etc.)), or selection of a different ML algorithm. Once the revision plan is decided upon at step S4, the method returns to steps S1 to S3.

If it is determined at S3 that the results are satisfactory, the method proceeds to step S5. At step S5, an expanded risk assessment may be performed on the AI system, and on the model. In particular, the risk assessment includes identification of additional risks that were not or could not have been considered beforehand. This step is likely performed by the expert team, apart from the at least one controller. The method also includes step S6, where an eXplainable Artificial Intelligence (XAI) component is used to analyze the model trained at step S1 and run at step S2. Based on the results of step S6, a further determination is made at step S7 either to revise the model (return to step S4) or to proceed to step S8.

Again referring to the example provided above, the action of step S6 may involve generating values for individual features of a feature vector (e.g., a histogram of oriented gradients (HOG) feature vector, or feature set) used by the classification model in classifying the intervention. According to one embodiment, SHapely Additive exPlanations (SHAP) are applied to generate a value for an individual feature of the feature set according to a contribution of the individual feature to the classification performed by the classification model. Further, the values generated by the SHAP may be used to generate a graphical representation (as discussed below), which graphical representation can then be assessed to determine if further revisions are required, or if audit evidence can be provided. The use of SHAP may be suggested by its theoretically sound foundation, its model-agnostic nature, and the characteristics of the ML model to be validated (e.g., use of a Random Forest algorithm).

As one example, the graphical representation may indicate that the AI system is classifying an intervention as critical solely because of the presence of a second hand inserted into an isolator. In the training data set, one-handed interventions were used to depict critical interventions, while two-handed interventions mostly depicted non-critical interventions. Respective quantitative performance metrics remained relatively good, but the graphical representation of the features contributing to the classification identified this feature as contributing to the classification and meriting a further determination.

If it is determined that the model should be revised, the model may require additional training. For example, the at least one controller may be configured to train the classification model with additional image frames of interventions assigned to at least two different classes if it is determined based on the graphical representation that the model provides insufficient performance. In the case of the above example involving one-handed and two-handed interventions, the determination was made to (re)train the classification ML model with additional image frames of new interventions (labelled with two or more classes) to remove this bias. Other revisions may also be performed, as explained above.

If it is determined at step S7 that no limitation is present, and no revision is required, the method may provide audit evidence at step S8, supporting the fact that the operation of the AI system is reliable. For example, if it is determined based on the graphical representation of the values for the features contributing to classification that the model requires no additional training, the at least one controller may generate audit evidence that may be used to establish that the AI system is reliable. The evidence may include documentation of the performance of the method as explained above, including the determinations made and any revision of the model that may occur.

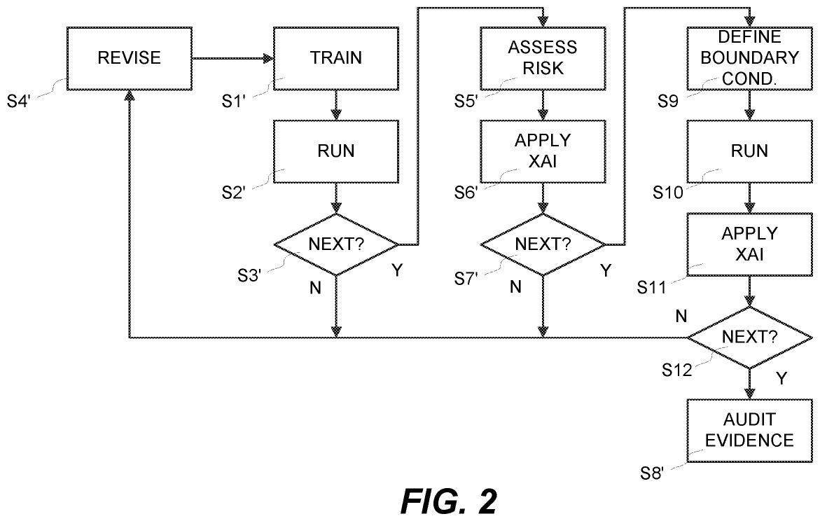

Another embodiment of a method of validating is illustrated in FIG. 2. The method of FIG. 2 shares several steps with the method of FIG. 1. Consequently, those steps shared in common with FIG. 1 are illustrated in FIG. 2, except that the similar steps in FIG. 2 are indicated with a prime (e.g., step S1′). Because these steps have been discussed in greater detail above, the discussion will not be repeated here, but those steps new to the method of FIG. 2 will be discussed below.

If the determination is made at step S7′ to continue (instead of revising the ML model at step S4′), the method of FIG. 2 continues to step S9. At step S9, boundary conditions may be determined for the model, which boundary conditions may represent possible, but unlikely, circumstances that may be presented to the AI system in use. At step S10, the model may be used to perform a classification of a boundary condition intervention using the classification model. That is, a test data set is compiled. The test data set may include real process data, but synthetic data may be acceptable if real process data is not available or it is unsafe to obtain it. At step S11, the XAI (e.g., SHAP) may again be applied to generate feature contribution values to be visualized as results of the classifications of the boundary condition intervention(s).

Again returning to the example, the actions at step S11 may include using a value assigned using the classification model, the value corresponding to a contribution of a feature to the classification of the boundary condition intervention, and generate a graphical representation of the values for the features contributing to classification of the boundary condition interventions. Here as well, SHapely Additive exPlanations (SHAP) may be applied to generate feature contribution values for the graphical representation, as explained in detail below. According to this embodiment, the features may be a histogram of oriented gradients (HOG) features.

If it is determined based on the XAI applied (e.g., the graphical representation of the values for the features contributing to classification of the boundary condition interventions) that the model requires additional training, the method may return to the step S4′. For example, the revision may include training the classification model with additional image frames of interventions assigned to at least two different classes. If it is determined at step S12 that no limitation is present, and no revision is required, the method may provide audit evidence at step S8′, supporting the fact that the operation of the AI system is reliable.

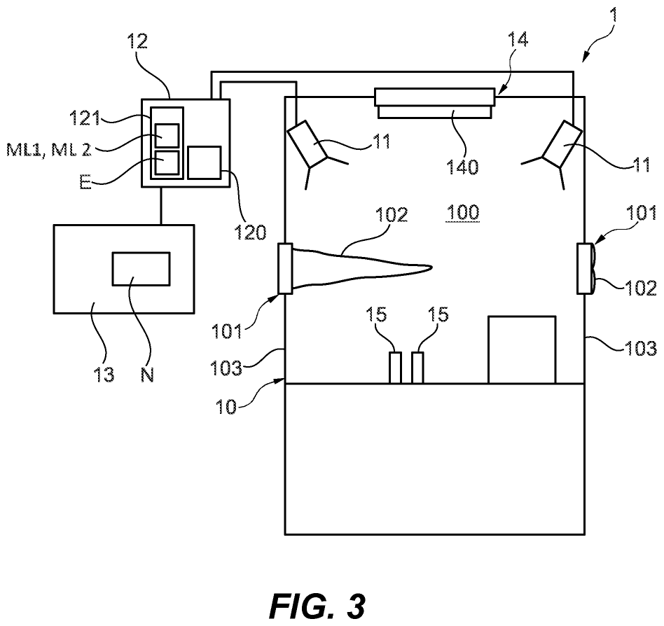

Having described the method and system for validating a system and method for monitoring pharmaceutical operations in general terms, the system and method for monitoring is now discussed in detail with reference to FIGS. 3-6. The system and method not only includes a classification model, but this classification model, which is the subject of the validation, may rely upon another model for input. Moreover, the role of the XAI in providing a graphical representation of the contribution of the features of the classification model is also illustrated and explained.

The embodiment of the system and method for validating a system and method for monitoring critical pharmaceutical operations is not limited to the embodiment of the system and method for monitoring described herein, however. The embodiment of the system and method for monitoring is provided to permit the system and method for validating to be described in additional detail, and to appreciate, in part, the scope of the systems and method for monitoring that may be validated.

Thus, FIG. 3 shows a system 1 for monitoring critical pharmaceutical operations in an aseptic interior space 100. That is, monitoring operations carried out by instruments to perform the production of medicine or medical nutrition of the like, for example in an enclosure.

The system 1 comprises an enclosure 10 defining the interior space 100, generally one or more cameras 11, here two cameras 11, are installed so as to record image frames of the interior space 100. Here, the cameras are arranged at an upper area of the enclosure 10 (inside the interior space 100) facing downwards.

The enclosure 10 comprises walls 103. The walls 103 delimit the interior space 100. The walls 103 isolate the interior space 100 from the surrounding environment.

Inside the interior space 100 various items are arranged, such as vials 15. The enclosure 10 is equipped with instruments to perform critical pharmaceutical operations, e.g., the production of medicine or medical nutrition or the like.

The system 1 further comprises glove ports 101. The enclosure 10 is a glove box. Each of the glove ports 101 is mounted in one of the walls 103 of the enclosure 10. The walls 103 may be glass panels. Each glove port 101 comprises a glove 102. An operator may insert a hand into one or more of the gloves 102. For illustrative purposes, one glove 102 (the left one in FIG. 3) is shown in a state inside the interior space 100, while the other glove 102 (the right one in FIG. 3) is shown in a state not inserted into the interior space 100. The glove ports 101 and the gloves 102 are within the field of view of each of the cameras 11 (generally, of at least one of the cameras 11).

The system 1 comprises a ventilation 14. The ventilation 14 comprises an air filter 140. The air filter 140 is adapted to filter air supplied to the enclosure. The air filter 140 is adapted to filter dust and germs from the air. The enclosure 10 of FIG. 3 is an isolator. An isolator is a type of clean air device that creates an almost complete separation between a product and production equipment, personnel, and surrounding environment. Operators who operate a production line can take actions inside isolators via the glove ports 101 in order to perform tasks required for the production process (required interventions, e.g., sedimentation disk changes) or to perform manipulations of objects/devices to maintain the production process (maintenance interventions, e.g., removing empty vials that fell off a conveyor). These interventions have to be documented and further measures have to be taken depending on the parameters (position, time, duration and/or class (e.g., critical or non-critical intervention)) of the interventions performed (e.g., rejecting one or more already filled vials due to the detection of critical interventions). Notably, however, aseptic filling is not limited to isolators.

Aseptic filling and other critical pharmaceutic operations can also be performed in specially designed clean rooms (class A with background cleanroom class B) or in RABS (restricted access barrier system) installations. Those impose a much higher risk to the product compared to isolator operations and interventions must be even closer monitored but are still widely used in pharma production.

Further, the system 1 comprises a controller 12 configured to receive the image frames recorded by the cameras 11, to analyze the image frames to detect an event captured by one or more of the image frames using a first model ML1. To perform a classification of an intervention captured by one or more of the image frames the controller uses a second model ML 2 (the classification model), the second model ML2 being trained with image frames of interventions assigned to at least two different classes, and to provide a notification N indicating one of the at least two different classes based on the classification.

The event may be an intervention, e.g., an intervention of at least one operator. For example, the intervention is an action performed inside the interior space. The intervention may be performed via one or more of the glove ports.

For example, for the at least two different classes it is distinguished between critical and non-critical interventions. Critical interventions comprise at least one critical image frame. The single image frames during one intervention are assigned to critical frames and non-critical frames.

To detect and classify events and/or interventions within the interior space 100, the controller 12 is connected to the cameras 11 so as to receive a video stream of image frames from each of the cameras 11. The controller 12 comprises a processor 120 and a memory 121. The memory 121 stores executable code E and the first and second model. The notification N provided by the controller 12 is displayed on a display device 13.

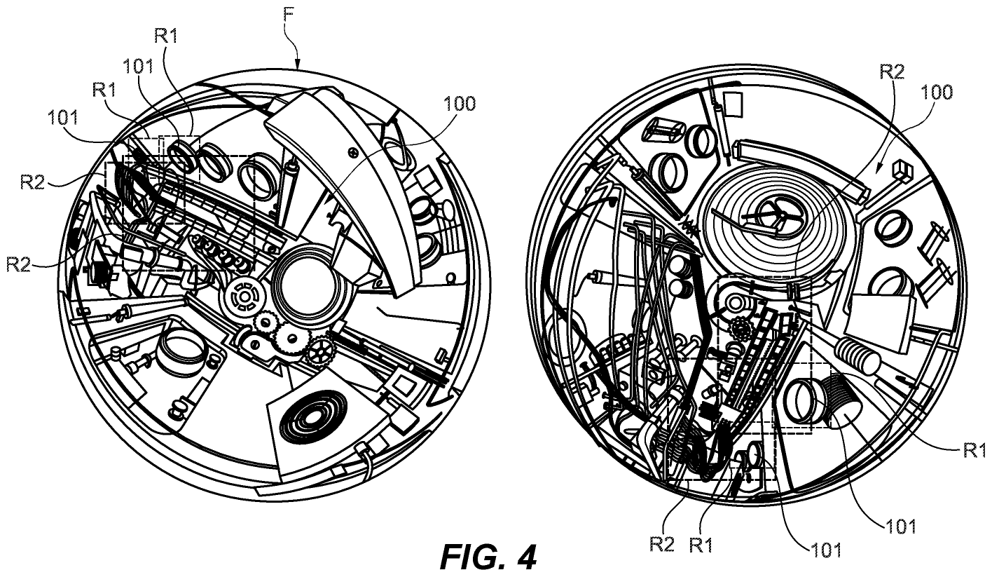

FIG. 4 shows a combined image frame F comprising an image frame of each of the cameras 11. This allows a simplified processing, but it is worth noting that the image frames of both cameras 11 could also be processed independently in parallel.

The viewing angle of each of the cameras 11 is fixed relative to the enclosure 10. As an example, two of the glove ports 101 are monitored. It will be appreciated, however, that more than two, e.g., all glove ports 101 of the system 1 may be monitored.

At fixed positions in the image frame F, pre-defined first regions R1 at the monitored glove ports 101 are defined. Here, each of the pre-defined first regions R1 includes one of the glove ports 101. The pre-defined first regions R1 are box shaped but could alternatively have another shape. At further fixed positions in the image frame F, pre-defined second regions R2 at the monitored glove ports 101 are defined. Here, each of the pre-defined second regions R2 includes at least a part of one or more of the glove ports 101, although R2 may in fact include no glove port at all. The pre-defined second regions R2 are box shaped but could alternatively have another shape. For each monitored glove port 101 a respective pre-defined first region R1 and a respective pre-defined second region R2 may be defined. Each pre-defined second region R2 may include a larger area than the corresponding pre-defined first region R1, although this will depend on factors such as lens distortion and/or position of the glove port in the isolator relative to the camera.

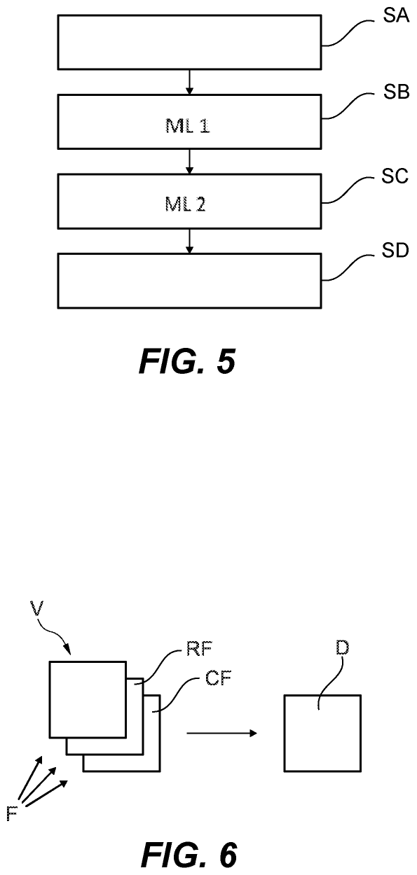

When executed by the processor 120, the executable code E stored in the memory 121 causes the processor 120 to perform the method of FIG. 5. In the method, the following steps are performed:

Step SA: Receiving, by the controller 12, image frames F recorded by the at least one camera 11, the at least one camera 11 being installed so as to record the image frames F of the interior 10 space 100 defined by the enclosure 10. The processing of the image frames is performed in a two-stage computer vision algorithm, comprising steps SB and SC.

Step SB: Analyzing, by the controller 12, the image frames F to detect an event captured in one or more of the image frames F. To detect the event, the pre-defined first regions R1 (see FIG. 4) are analyzed by a trained machine learning first model (ML 1) for event detection (event-detection model). The trained event-detection model is stored in the memory 121. For this analysis, the controller 12 calculates a histogram of oriented gradients, HOG, for the respective pre-defined first regions R1, which is provided to the event-detection model (first model) as input. The event-detection model determines a classification result which is either positive (event detected) or negative (no event detected). The event-detection model is trained using training image frames (in particular, with the respective HOGs) with positive and negative classifications (i.e., results in a binary classifier). As an example, an intervention may be defined as being imminent if one of the gloves 102 is inside the enclosure 10. Correspondingly, when no glove 102 is inside the enclosure 10, the respective image frame F may be defined as not depicting an intervention. Optionally, different types of events (particularly interventions) may be detected. In the present example, a Random Forest algorithm is used as the event-detection model. Optionally, an event may be detected if at least one frame (or, alternatively, at least another threshold number, e.g., 2, 3 or 4 of consecutive frames) are classified as showing an event are detected.

Step SC: Performing, by the controller 12, a classification of the detected intervention captured by the one or more of the image frames F classified as showing an event, using a second model ML 2 as classification model. As soon as an event is detected starting with a given image frame F, the last image frame F before that has not been classified as showing an event is defined as a reference frame RF. In step SC, a current frame CF currently being classified, and the reference frame RF are used to compute a difference image D, see FIG. 6. This difference image D is used for the analysis. Here, the difference image D is used to compute HOG features which are then input to the classification model ML 2. Once a single image frame F is detected as critical, the whole intervention is considered critical. Critical sequences also contain non-critical image frames F, typically in the beginning and at the end, and at least one critical image frame F. The end of an event is determined when a threshold number (e.g., 1, 2, 3 or 4) of consecutive image frames F are classified as not showing an event. Each event has a corresponding reference frame RF. That is, for every newly detected event, a respective reference frame RF is determined.

In the present example, step SC is only performed for image frames F after an event is detected in step SB. The second model ML 2 is trained with image frames F of interventions (in general: actions) assigned to at least two different classes, here: critical or non-critical. The second model ML 2 is trained using training image frames (in particular, with the respective HOGs) from critical and non-critical interventions (i.e., yields another binary classifier). In the present example, another binary Random Forest algorithm is used as the second model ML 2. For example, a critical image frame may be one where the glove 102 touches a given surface or is too close to a given object. To name few examples, an intervention may be a part of media filling processes, adjusting filling needles or a change of sedimentation disks.

Optionally, additional parameters are used to calculate the probability that the intervention is critical, e.g., the duration of the intervention.

Steps SB and SC are performed for each glove port 101 individually. Thus, more than one event may be detected simultaneously. For example, one (e.g., non-critical) intervention at one glove port 101 may be performed at the same time as another (e.g., critical) intervention at another glove port 101.

The training data may have been classified manually or using other reliable methods. Another set of pre-classified image frames may be used as test data set to test the performance of the event-detection model and/or the classification model.

Step SD: Providing, by the controller 12, a notification N indicating one of the at least two different classes based on the classification. Optionally, the system 1 and method record all recognized interventions (more general: events) and parameters thereof (e.g., date and time, duration, type of intervention etc.). Then the operator may be notified of upcoming required interventions. The record may be used for quality control and assurance and/or to trigger corrective actions depending on the recognized interventions.

The method is performed in real-time (alternatively, post-hoc) on a video stream V (see FIG. 6) comprising a sequence of image frames F. The frame rate may be, e.g., between 5 and 20 frames per second, particularly 10 frames per second.

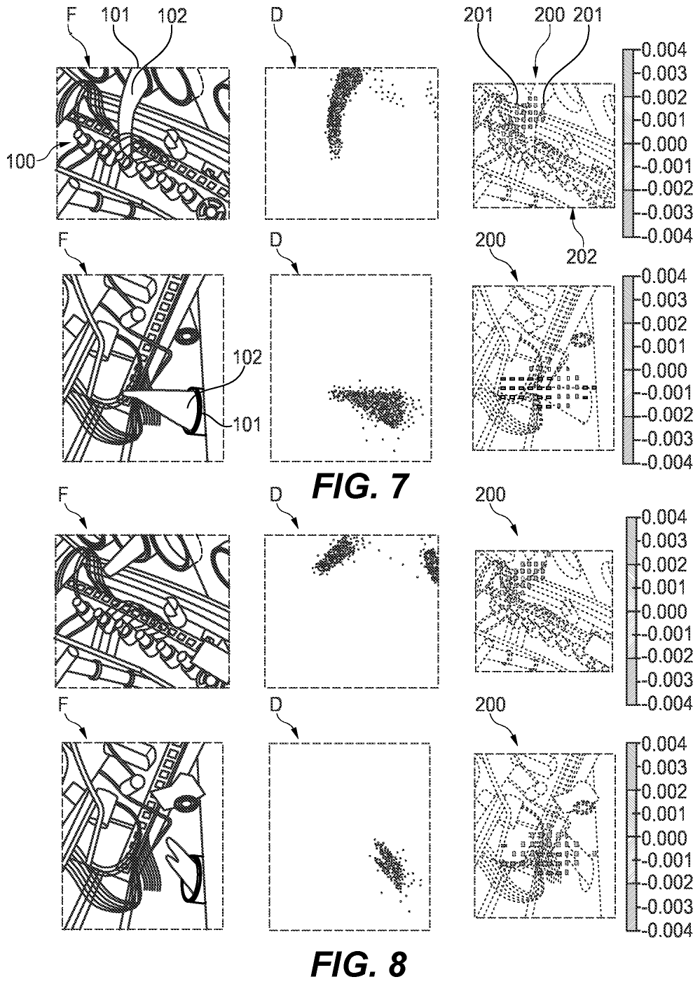

Turning now to FIGS. 7 and 8 the functionality of the classification will be described in more detail. In particular, the combination of the classification model and the XAI is illustrated.

FIG. 7 shows on the left image frames F of the two cameras 11 showing a critical intervention. In the middle, corresponding difference images D are shown. On the right, graphical representations 202 comprising the corresponding HOGs 200 are shown. Each HOG 200 comprises a plurality of HOG features 201. Each HOG feature 201 is assigned, by means of the second classification model ML 2, a value which corresponds to its contribution to the model's decision.

FIG. 8 shows the same as FIG. 7, just for a non-critical intervention.

To allow a user to gain insights into why the Random Forest classified image frames F as critical or non-critical, the graphical representations 202 are displayed, e.g., on display device 13. Here, the HOG features 201 may be overlaid the respective image frame F (optionally shaded). More specifically, SHapely Additive exPlanations (SHAP) are applied to visualize the contribution of individual HOG features 201 in an image. FIGS. 7 and 8 show positive SHAP values (towards green, contribute to a non-critical decision) and negative SHAP values (towards red, contribute to a critical decision).

Thus, while usually an ML model in an analysis may be regarded as a black box, here it is possible to directly visualize the data which is the basis for the decision of the second model ML 2. This allows more reliable results and simplified certification in many fields of application, and may be integrated into a method and system of validation as explained with reference to FIG. 1 or FIG. 2.

The basic idea of HOG is that based on the gradients (intensity differences of neighboring pixels) a robust color and size independent objective description of the image content is obtained. The entire image section used for classification (second regions R2) is scaled to a fixed size and divided into 8×8 pixel cells in which a histogram is formed over the 9 main directions (0-360°). That is, each cell is described by a 9-bin histogram. Then, these features are normalized, and the histograms are lined up. This then results in a feature vector, where each number in the vector is called a feature.

While above the event has been described as an intervention using a glove port 101, it will be appreciated that the same algorithm may be applied for other kinds of events. Indeed, the system 1 does not necessarily have to comprise glove ports 101 at all.

Notably, in addition to the one or more cameras 11 other sensor types may be used to provide input to the analysis described above, e.g., LiDAR (Light Detection and Ranging) sensors.

Although the preceding text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

What is claimed is:

1. A system for validating a system for monitoring pharmaceutical operations, the system comprising:
at least one controller configured to:
perform a classification of an intervention captured by one or more image frames using a classification model, the classification model being trained with image frames of interventions assigned to at least two different classes,
assign a value to an individual feature of a feature set associated with the classification, the value corresponding to a contribution of the individual feature to the classification, and
generate a graphical representation of the values for the features contributing to the classification of the interventions.

2. The system according to claim 1, wherein the at least one controller is configured to train the classification model with additional image frames of interventions assigned to at least two different classes if it is determined based on the graphical representation that the model requires additional training.

3. The system according to claim 1, wherein the at least one controller is configured to provide audit evidence if it is determined based on the graphical representation that the classification model requires no revision.

4. The system according to claim 1, wherein SHapely Additive exPlanations (SHAP) are applied to generate the value for the individual feature, the value corresponding to a contribution of the individual feature to the classification.

5. The system according to claim 4, wherein the feature set is a histogram of oriented gradients (HOG) feature set.

6. The system according to claim 1, wherein the system for monitoring pharmaceutical operations comprises an enclosure defining an interior space, and at least one camera configured to record image frames of the interior space,
the at least one controller configured to:
receive the image frames recorded by the at least one camera,
perform a detection of an event in one or more image frames using an event-detection model, the event-detection model being trained with image frames assigned to at least two different classes, and
perform the classification of an intervention on the one or more image frames if classified as showing an event.

7. The system according to claim 1, wherein the at least one controller is configured to:
perform a classification of a boundary condition intervention using the classification model if it is determined based on the graphical representation that the model does not require revision,
assign a value to an individual feature of a feature set associated with the classification, the value corresponding to a contribution of the individual feature to the classification,
generate a graphical representation of the values for the features contributing to classification, and
train the classification model with additional image frames of interventions assigned to at least two different classes if it is determined based on the graphical representation of the values for the features contributing to classification that the model requires additional training.

8. The system according to claim 7, wherein the at least one controller is configured to provide audit evidence if it is determined based on the graphical representation of the values for the features contributing to classification that the classification model requires no revision.

9. The system according to claim 7, wherein SHapely Additive exPlanations (SHAP) are applied to generate the value for the individual feature, the value corresponding to a contribution of the individual feature to the classification.

10. The system according to claim 9, wherein the feature set is a histogram of oriented gradients (HOG) feature set.

11. A method for validating a method for monitoring pharmaceutical operations, comprising
performing a classification of an intervention captured by one or more image frames using a classification model, the classification model being trained with image frames of interventions assigned to at least two different classes,
assigning a value to an individual feature of a feature set associated with the classification, the value corresponding to a contribution of the individual feature to the classification, and
generating a graphical representation of the values for the features contributing to the classification of the interventions.

12. The method according to claim 11, further comprising training the classification model with additional image frames of interventions assigned to at least two different classes if it is determined based on the graphical representation that the model requires additional training.

13. The method according to claim 11, further comprising providing audit evidence if it is determined based on the graphical representation that the classification model requires no revision.

14. The method according to claim 11, wherein SHapely Additive exPlanations (SHAP) are applied to generate the value for the individual feature, the value corresponding to a contribution of the individual feature to the classification.

15. The method according to claim 14, wherein the feature set is a histogram of oriented gradients (HOG) feature set.

16. The method according to claim 11, further comprising:
receiving image frames recorded by at least one camera configured to record the image frames of an interior space defined by an enclosure,
performing a detection of an event in one or more image frames using an event-detection model, the event-detection model being trained with image frames assigned to at least two different classes, and
performing the classification of an intervention on the one or more image frames if classified as showing an event.

17. The method according to claim 11, further comprising:
performing a classification of a boundary condition intervention using the classification model if it is determined based on the graphical representation that the model does not require revision,
assigning a value to an individual feature of a feature set associated with the classification, the value corresponding to a contribution of the individual feature to the classification,
generating a graphical representation of the values for the features contributing to classification, and
training the classification model with additional image frames of interventions assigned to at least two different classes if it is determined based on the graphical representation of the values for the features contributing to classification that the model requires additional training.

18. The method according to claim 17, further comprising providing audit evidence if it is determined based on the graphical representation of the values for the features contributing to classification that the classification model requires no revision.

19. The method according to claim 17, wherein SHapely Additive exPlanations (SHAP) are applied to generate the value for the individual feature, the value corresponding to a contribution of the individual feature to the classification.

20. The method according to claim 19, wherein the feature set is a histogram of oriented gradients (HOG) feature set.
